(19) United States
(12) Patent Application Publication
(10) Pub. No.: US 2026/0054868 A1
(43) Pub. Date: Feb. 26, 2026

(54) SYSTEM FOR PROCESSING PHARMACEUTICAL CONTAINERS AND METHOD FOR OPERATING SUCH A SYSTEM

(71) Applicant: Bausch + Stroebel SE + Co. KG, Ilshofen (DE)

(72) Inventors: Nico Butzer, Ilshofen (DE); Oliver Lang, Ilshofen (DE); Philip Messerschmidt, Ilshofen (DE); Benjamin Spang, Ilshofen (DE)

(21) Appl. No.: 19/372,927

(22) Filed: Oct. 29, 2025

Related U.S. Application Data
(63) Continuation of application No. PCT/EP2024/060322, filed on Apr. 16, 2024.

(30) Foreign Application Priority Data
Apr. 30, 2023 (DE) 10 2023 001 783.3
May 3, 2023 (DE) 10 2023 111 480.8

(51) Int. Cl. B65B 3/00; A61L 2/07; A61L 2/18; B25J 21/00; B65B 3/26; B65B 7/28; B65B 43/46; B65B 55/02; B65B 57/00; B65B 65/00

(52) U.S. Cl. CPC B65B 3/003; A61L 2/07; A61L 2/186; B25J 21/005; B65B 3/26; B65B 7/28; B65B 43/46; B65B 55/027; B65B 57/00; B65B 65/006; A61L 2202/14; A61L 2202/182; A61L 2202/23

(57) ABSTRACT

A system and method for processing pharmaceutical containers includes at least one production facility for filling and closing the containers in at least one processing room and at least one preparation device which is or can be arranged spatially remote from the at least one production facility. At least one transportable container device can be docked to the production facility for introducing pharmaceutical production material which comprises the containers and closure elements. The container device can be docked to the preparation device to receive the production material. A metering device can be docked to the production facility in order to insert a filling needle and a fluid conduit.

### CROSS-REFERENCE TO RELATED APPLICATIONS

This application is a continuation of International Application No. PCT/EP2024/060322, filed on Apr. 16, 2024, and claims priority to German Application No. 10 2023 001 783.3, filed on Apr. 30, 2023, and to German Application No. 10 2023 111 480.8, filed on May 3, 2023. The contents of International Application No. PCT/EP2024/060322, German Application No. 10 2023 001 783.3 and German Application No. 10 2023 111 480.8 are incorporated by reference herein in their entireties.

### FIELD

The present disclosure relates to a system for processing pharmaceutical containers which comprises a production facility for filling and closing the containers in a processing room, the production facility comprising a filling station for filling the containers with a pharmaceutical product and a closing station for closing the containers with closure elements.

The present disclosure further relates to a method for operating such a system.

### BACKGROUND

In a production facility of the type described above, pharmaceutical containers are filled with, for example, a liquid product. The containers are, for example, vials, syringes, cartridges or ampoules. The containers can be stable and stand independently on a set-down surface. Alternatively, they can be non-stable containers that have to be held by means of a holding device.

A high-quality cleanroom is usually required in the production facility, especially for small and very-small series, particularly in the “high potent” field and/or in the field of toxic products. A cleanroom class of type GMP A (GMP: good manufacturing practice) is desirable.

Before the actual production, conventional facilities require extensive preparatory measures, which include, for example: cleaning the facility, installing components that do not come into contact with the product to be filled and that do not need to be sterilized, closing the facility, decontamination, in particular with H₂O₂ steam, to create a sufficient cleanroom class (e.g., Class A) required for production, parallel sterilization of product-contacting components, introduction of these components into the processing room by a user via glove ports (aseptic setup). Once preparation is completed, the product is filled, wherein after the filling process, the facility must be cleaned again even before any closures are opened, for example in the case of toxic production.

In conventional production facilities, the above steps not only require a lot of time, but the production facility is also subject to the risk of possible unwanted contamination. For example, certain components of the production facility are difficult to sterilize and/or difficult to integrate into the production facility due to their size. These include, for example, sorting apparatuses with pots for closure elements and their sorting tracks. As a result, in the case of a large number of components, the entire production cycle is determined in view of the risk of contamination from the weakest link in the chain. Any malfunctions must be resolved manually by the user via glove ports. Subsequently, production approval is required; if this is not granted, in the worst-case scenario, an entire batch will have to be discarded.

### SUMMARY

The object of the present disclosure is to provide a system for processing pharmaceutical containers and a method for operating such a system in which hygienic requirements for production can be met in a more reliable manner.

This object is achieved by a system in accordance with the present disclosure for processing pharmaceutical containers, comprising:
at least one production facility for filling and closing the containers, which comprises, in at least one processing room, a filling station for filling the containers with a pharmaceutical product and a closing station for closing the containers with closure elements, wherein the at least one production facility comprises a controllable robotic handling device which is arranged in at least one processing room, is configured for handling pharmaceutical production material comprising containers and/or closure elements received in a predefined arrangement in receiving bodies, and which comprises or forms at least one connection device for docking an external transportable container device and/or an external transportable metering device;
at least one preparation device which is or can be arranged spatially remote from the at least one production facility and comprises or forms at least one work space and at least one connection device for docking the container device, wherein pharmaceutical production material can be introduced into the at least one work space, which production material comprises containers to be processed and/or closure elements, wherein the containers and/or the closure elements can be inserted into the receiving body in a predefined arrangement in the at least one work space;
at least one transportable container device, by means of which the containers received in the receiving body and/or the closure elements received in the receiving body can be brought directly or indirectly from the at least one preparation device to the at least one production facility and, when the container device is docked to the at least one production facility, can be introduced into the at least one processing room by means of the handling device;
at least one metering device comprising, in a receiving space, pharmaceutical metering material comprising a fluid conduit and a filling needle arranged thereon, which can be introduced into the at least one processing room by means of the handling device when the metering device is docked to the at least one production facility.

The present disclosure is based on the consideration that risks and/or disturbances in production can be reduced and/or preferably largely avoided if the at least one production facility, which to a certain degree carries out the “core of production”, can be decoupled from preparatory actions. For filling the product and at least a closure of a container via the closure element, preferably pharmaceutical sealing, the production facility comprises, in the at least one processing room, a filling station and a closing station. Containers and closure elements can preferably be supplied via respective receiving bodies and handled by the robotic handling device. For this purpose, the handling device can be provided, in particular, with information about the position of a particular receiving body at which a container or a closure element is arranged, as well as how these are to be moved in the processing room. Preparatory activities during production can be carried out in the at least one preparation device. Preferably, it is possible in particular to transfer the containers and/or the closure elements into respective receiving bodies in the defined arrangement required for processing in the production facility. The transfer takes place via the at least one container device, which is first docked to the preparation device in order to receive containers or closure elements, whereupon the container device can subsequently be docked to the at least one production facility in order to remove the containers or closure elements. In accordance with the present disclosure, a portable metering device is further provided which can comprise, in a work space, metering material which can in particular comprise a fluid conduit and a filling needle arranged thereon. When the metering device is docked to the production facility, the filling needle and the fluid conduit, the latter at least partially, can be introduced into the processing room and inserted at the filling station.

It can be provided that a holding part of the metering device, on which part the filling needle and/or the fluid conduit are held and which is transferred from the receiving space of the docked metering device into the processing room, forms part of the filling station.

From the above explanations, it can be seen that, in accordance with the present disclosure, by decoupling the actual production process from preparatory steps, hygienic requirements can be met in a simpler and more reliable manner. Preparatory actions such as decontamination and/or sterilization can be carried out externally to the at least one production facility and thus optimized with regard to the preparation process. In a corresponding manner, the actual production process can be optimized with regard to its implementation in the at least one production facility. In this way, the filling and closing of containers can be carried out with a lower risk of disruption in the production facility itself, in particular by using the robotically controllable handling device. Preferably, the number of components in the processing room can be reduced, which lowers the risk of disruption and thereby reduces the risk of undesirably open but already filled containers.

The at least one container device and the at least one metering device can be docked to at least one connection device of the production facility. In the present case, this can be understood in particular to mean that the system comprises a connection system in which components of the container device and the metering device interact with components of the production facility. For example, corresponding ports are used. In particular, the use of a so-called RTP (rapid transfer port) is conceivable. In this case, the connection device can comprise an insertion opening in the production facility and a closing element arranged thereon for selectively opening and closing the insertion opening. A removal opening can be arranged on the container device or the metering device, as can a corresponding closing element by means of which the removal opening can be opened or closed. When the container device or metering device is docked, the removal opening and the insertion opening can communicate with each other so that the contents of the container device or metering device can be introduced into the processing room. If an RTP is provided, an alpha port can be provided on the production facility and a beta port can be provided on the container device or the metering device.

The at least one connection device can, for example, be arranged directly on a wall arrangement of the at least one processing room, to which wall arrangement the container device or the metering device can be docked. The production material and/or the filling conduit and the filling needle can be introduced directly into the processing room by means of the handling device.

The production facility can comprise an optional in-coupling cell having at least one connection device for the container device and/or for the metering device. The production material and/or the filling conduit and the filling needle can be introduced into the processing room through the in-coupling cell by means of the handling device, for example.

In accordance with the above, production material and/or metering material can be introduced directly or indirectly, through the optional in-coupling cell, into the at least one processing room.

Typically, each receiving body will receive a plurality of containers or a plurality of closure elements. However, the present disclosure is not limited to this. It can be the case that each receiving body receives only one container or only one closure element.

For example, containers and closure elements are received in separate receiving bodies. However, it can be provided that at least one container and at least one closure element are received in one receiving body.

The receiving bodies can be transferred into the at least one processing room by means of the handling device, for example together with containers or closure elements. Alternatively, it is conceivable that the receiving bodies remain in the container device and only the containers or closure elements are transferred into the processing room.

Favorably, the system comprises at least one control device for controlling the at least one production facility and/or the at least one preparation device. It can be provided that each production facility and/or each preparation device comprises an independent control device. Two or more control devices can be coupled to each other via information technology. The control device of the production facility is provided in particular to control the robotic handling device, the filling station and/or the closing station.

The system can comprise at least one data processing device for controlling and/or regulating the system. The data processing device can be located locally at the site of the system or spatially remote from it. The data processing device can be formed by one or more servers, which are located, for example, in a cloud.

The data processing device can comprise or form the at least one control device, or vice versa.

Preferably, the system is characterized by a cleanliness class of the at least one processing room in accordance with GMP Type A. If the containers in the production facility have been pharmaceutically sealed in a processing room, a subsequent processing room can have a lower cleanliness class, for example in accordance with GMP Type C.

If the production facility has a consistent cleanliness class throughout, in particular of type GMP A, in at least one processing room and, if present, an optional in-coupling cell and/or out-coupling cell, it is possible in particular to implement a truly closed isolator device in the production facility, as described in Annex 1, “Manufacture of Sterile Medicinal Products” of August 2022 to the EU Guidelines for Good Manufacturing Practice (Annex 1 “Manufacture of Sterile Medicinal Products” to the “Guidelines for Good Manufacturing Practice for Medicinal Products for Human and Veterinary Use”). Accordingly, the production facility preferably comprises or forms a closed isolator device in accordance with Annex 1, “Manufacture of Sterile Medicinal Products”, to the EU Guidelines for Good Manufacturing Practice of August 2022.

Favorably, the system is characterized by a cleanliness class of the at least one work space in accordance with GMP Type C or better. For example, it can be that a work space has a cleanliness class of type GMP A. This is, for example, the work space from which the production material is transferred to the container device, which is docked to the preparation device.

Favorably, the system is characterized by a cleanliness class of the receiving space of the metering device of type GMP A. The same applies to the interior of the container device when docking to the at least one production facility.

It can be advantageous if the system comprises a separate sterilization device which is, for example, arranged spatially separate from the at least one production facility and the at least one preparation device. The sterilization device can in particular be an autoclave device. The sterilization device is preferably designed to sterilize objects received in the at least one container device and/or the at least one metering device. This provides, for example, the possibility of carrying out terminal sterilization after filling the at least one container device and/or the at least one metering device. This proves to be advantageous, for example, in the case where the preparation device and thus the objects do not (yet) have the required cleanliness when introduced into the container device and/or the metering device. Terminal sterilization allows the container device and/or the metering device to be upgraded to a higher cleanliness class, for example type GMP A.

The at least one preparation device can, for example, comprise an insertion opening via which the production material can be introduced into the work space. For example, closure elements are inserted into bags, which are sorted in the production device and transferred into the receiving bodies. Alternatively or additionally, for example, secondary packaging materials are introduced with the containers, which represent primary packaging materials, into the preparation device via the insertion opening, unpacked therein, and the containers are removed from the secondary packaging materials.

The at least one preparation device comprises, for example, a removal opening in the connection device, via which the production material is transferable into the container device. In a manner corresponding to that already explained in connection with the at least one production facility, a connection system is preferably formed by means of the preparation device and the container device and/or the metering device, with closing elements at the removal opening in the preparation device and at the container device and/or the metering device. It is favorable to use an RTP with an alpha port on the preparation device and a beta port on the container device and/or on the metering device.

The at least one production device can comprise a disposal opening for waste. Furthermore, the work space can contain a disposal station, for example, via which waste can be disposed of during preparatory actions.

With regard to required and/or desired degrees of cleanliness, it can be advantageous if the at least one preparation device comprises or forms at least one of the following:
a pharmaceutical isolator device which can favorably provide a cleanliness class of type GMP A in the work space;
a decontamination device for providing a defined decontamination atmosphere in the at least one work space, in particular by means of H₂O₂, and/or a connecting element for connecting an external decontamination device to the preparation device. For example, non-product-contact components that are to be transferred into the production facility can be decontaminated by means of the H₂O₂ atmosphere.

The system can comprise two or more preparation devices. The preparation devices can be configured identically or differently.

It can be provided that the system comprises a preparation device for providing the containers and a preparation device separate therefrom for providing closure elements. It is understood that, for example, the containers or the closure elements can be received and brought to the at least one production facility by means of different container devices.

The system can comprise, for example, a preparation device for providing culture media for introduction into the at least one production facility via the at least one container device, the production device preferably being separate from a production device for providing containers and/or closure elements. In the production facility, for example, culture media are used to monitor sterility. By means of a suitable production device, culture media, for example, are introduced into the work space in bags and, arranged in receiving bodies, are inserted into the container device, by means of which the culture media are brought to the at least one production facility.

The system can comprise, for example, a preparation device for providing format parts and/or machine parts for introduction into the at least one production facility via the at least one container device, the preparation device preferably being separate from a production device for providing containers and/or closure elements. Format parts, which are configured in particular to be container-specific or closure-element-specific, can also be prepared in this way outside the processing room of the production facility and then transported to the at least one production facility by means of the at least one container device. Format parts and machine parts can be sterilized and/or decontaminated in the production device or in a separate sterilization device.

It has already been mentioned that the at least one metering device can be docked to a production device. Accordingly, it can be advantageous if the system comprises a preparation device for providing the metering material in the work space, it being possible for the at least one metering device to be docked to the preparation device and the metering material being transferable into the receiving space. The metering material, in particular a filling conduit and a filling needle, can be prepared in this way, externally to the at least one production facility. In this case, sterilization and/or decontamination in the preparation device or a separate sterilization device is conceivable. For introduction into the metering device, the metering material can comprise, for example, a fluid conduit, a filling needle, a metering unit, a container for receiving the product and/or a filter element connected into the fluid conduit.

It can be provided that the at least one preparation device comprises two separate work spaces which are connected to one another via a transfer opening.

The transfer opening can be permanently open, for example if both work spaces have the same cleanliness class. If the work spaces have different cleanliness classes, an open transfer opening can be provided if there is a gas flow from the work space with the higher cleanliness class to the work space with the lower cleanliness class. Such a gas flow is preferred if, at the transfer opening, a closing element is present which is only temporarily open for the transfer. At the transfer opening an airlock device can be provided which can preferably be decontaminated by means of an H₂O₂ cycle.

In one of the two work spaces, for example, production material is prepared, in particular unpacked and/or decontaminated, the production material being introduced into the second work space via the transfer opening, and it being possible for the container device to be docked in the region of the second work space.

The preparation of production material, which comprises in particular containers, closure elements, culture media and/or format parts, can be carried out in two stages in such a preparation device. For example, the production material is introduced into the first work space, where it can be unpacked and subsequently sterilized and/or decontaminated. For this purpose, for example, a glove port for a user is provided in the first work space. The production material prepared in this way can be used in the second work space, for example in receiving bodies for transfer to the production facility. In the second work space, a glove port for the user and/or a robotically controllable handling device can be provided.

In accordance with the above, it can be favorable if the at least one preparation device comprises a controllable robotic handling device for moving the production material in the at least one work space and/or if the at least one preparation device has at least one glove port for a user in the at least one work space.

It can be provided that the system comprises, spatially separated from the at least one production facility and the at least one preparation device, a storage device for temporarily storing filled container devices and/or metering devices after filling at the at least one preparation device and before use at the at least one production facility. The storage device allows the container devices and/or metering devices to be temporarily stored so that production cycles can be processed efficiently. For example, the time of preparation of the container devices and/or metering devices can be decoupled from the time of production. Favorably, the sterile status of each container device and/or metering device is monitored. This is done, for example, by pressurizing the container device and/or the metering device and monitoring the positive pressure difference or by means of a pressure recorder. The container device and/or metering device can be brought to the production facility in a timely manner and production can begin.

The at least one metering device can comprise at least one of the following, preferably in the receiving space:
the fluid conduit and the filling needle;
a container for receiving the product to be filled;
a metering unit for conveying the product through the fluid conduit, for example configured as a pump unit;
a filter element connected into the fluid conduit, for example a sterile filter.

The system can comprise two or more production facilities, which can be configured identically or differently.

The at least one production facility can, for example, comprise two connection devices which are preferably configured differently from one another. Depending on the type of connection device involved, different types of devices can be docked. For example, the at least one container device can be docked to one connection device and the at least one metering device can be docked to the other connection device. The various connection devices differ in terms of their size, for example, but are otherwise configured to be functionally identical. For example, RTPs of different sizes are used. Different types of connection devices can simplify handling, for example, and prevent incorrect operation by ensuring that a device can only be docked to the correct connection device.

The filling station and the closing station can be arranged in the same processing room of the at least one production facility. Such a configuration can be used, for example, for processing syringes or cartridges that are considered pharmaceutically sealed with only one closure element.

In an exemplary embodiment, the at least one production facility comprises a further processing room and a further closing station, which is arranged in the further processing room, the processing rooms being connected to one another via a transfer opening. Accordingly, a production facility of this kind comprises two closing stations. In particular, the filling station and the closing station can be positioned in one processing room and the further closing station in the further processing room. Such a production facility is used, for example, to process vials which must first be closed with stoppers and then with crimp caps in order to be considered pharmaceutically sealed.

The transfer opening can be permanently open, for example if both processing rooms have the same cleanliness class. If the processing rooms have different cleanliness classes, an open transfer opening can be provided if there is a gas flow from the processing room with the higher cleanliness class to the processing room with the lower cleanliness class. Such a gas flow is preferred if, at the transfer opening, a closing element is present which is only temporarily open for the transfer. At the transfer opening an airlock device can be provided which can preferably be decontaminated by means of an H₂O₂ cycle.

In an exemplary embodiment, the at least one production facility can comprise a further processing room having a cleaning station, which are preferably connected to the at least one processing room via a transfer opening. Such a production facility is used, for example, in the processing of toxic products that require external cleaning after filling and sealing. The above applies to the transfer opening. The processing room can have a cleanliness class of GMP Type C.

In an exemplary embodiment, the at least one production facility can comprise a further processing room having a freeze-drying station, which is preferably connected to the at least one processing room via a transfer opening. Such a processing room is, for example, located downstream of a processing room with a filling station and closing station, in which vials are filled and closed with stoppers, and upstream of a further processing room in which the vials are provided with crimp caps. The above applies to the transfer opening.

In a preferred embodiment of the present disclosure, the at least one production facility is designed to be free of a glove port for the user. All manipulation and/or handling operations can favorably be carried out by means of the handling device in the at least one processing room.

It is conceivable that the at least one container device and/or the at least one metering device are transported and in particular carried by a user.

The at least one container device and/or the at least one metering device can be configured to be portable for transport.

However, it is favorable if the system comprises at least one transport device by means of which the container device is received or releasably receivable, and/or if the system comprises at least one transport device by means of which the metering device is received or releasably receivable, wherein the at least one transport device comprises a running gear for moving on a set-down surface. This facilitates the handling of the container device and the metering device.

Container devices and/or metering devices can be transported via the at least one transport device, in particular from the preparation device to the production facility, if necessary to the sterilization device and/or to the storage device. After filling and closing, the container devices can be received by means of the at least one transport device and supplied to further processing, for example inspection, labeling and/or further packaging.

In a preferred embodiment of the present disclosure, the at least one transport device is configured to be self-propelled and self-steering for autonomous transport of the at least one container device or the at least one metering device. This makes it possible to automate the system to a high degree. For example, transport devices are provided which can be controlled by means of the data processing device mentioned above. The transport devices comprise, in a storage unit for example, a map of the environment, on the basis of which transport paths can be planned, managed and/or processed.

Identification devices can be provided, by means of which transport devices can identify the container devices, metering devices, production devices and/or production facilities in order to ensure that production can proceed as intended so that each container device or metering device is transported to the production facility in good time and set up for the start of production and/or is picked up from the preparation device after setup.

The system favorably comprises a data processing device having a storage unit in which a production plan is stored, wherein at least one of the following can be controlled by the data processing device indirectly or directly based on the production plan: the at least one production facility, the at least one preparation device, the at least one transport device.

As mentioned at the outset, the present disclosure also relates to a method. A method in accordance with the present disclosure for operating a system of the type described above comprises:
docking at least one container device to the at least one production facility;
introducing into the processing room, by means of the at least one handling device, a receiving body which receives at least one container to be filled in a predefined arrangement;
introducing into the at least one processing room, by means of the handling device, a receiving body which receives at least one closure element in a predefined arrangement;
docking at least one metering device to the at least one production facility; introducing the filling needle and, at least partially, the filling conduit into the at least one processing room; and
filling and closing the at least one container.

The container device and the metering device can be docked simultaneously or one after the other, with the container device being docked before the metering device or vice versa.

The advantages already mentioned in connection with the explanation of the system in accordance with the present disclosure can also be achieved by performing the method. In this respect, reference is made to the above statements.

Advantageous exemplary embodiments of the method in accordance with the present disclosure follow from advantageous embodiments of the system in accordance with the present disclosure. Reference is made to the preceding statements.

### BRIEF DESCRIPTION OF THE DRAWINGS

The following description of preferred embodiments of the present disclosure serves in conjunction with the drawings to explain the present disclosure in more detail. In the figures:

FIG. 1: shows a schematic representation of the system in accordance with the present disclosure in a preferred embodiment;

FIG. 2: shows the system from FIG. 1 in a schematic representation with an exemplary layout of the arrangement of the components of the system;

FIG. 3: shows a production facility of the system, marked “A” in FIG. 2, in an enlarged, more detailed view;

FIG. 4: shows a further production facility of the system, marked “B” in FIG. 2, in an enlarged, more detailed view;

FIG. 5: shows a further production facility of the system, marked “C” in FIG. 2, in an enlarged, more detailed view;

FIG. 6: shows a further production facility of the system, marked “D” in FIG. 2, in an enlarged, more detailed view;

FIG. 7: shows a preparation device of the system, marked “E” in FIG. 2, in an enlarged, more detailed view;

FIG. 8: shows a further preparation device of the system, marked “F” in FIG. 2, in an enlarged, more detailed view;

FIG. 9: shows a further preparation device of the system, marked “G” in FIG. 2, in an enlarged, more detailed view;

FIG. 10: shows a further preparation device of the system, marked “H” in the system of FIG. 2, in an enlarged view of the detail at the top-left of FIG. 2;

FIG. 11: shows a schematic representation of a container device of the system;

FIG. 12: shows a schematic representation of a receiving body with containers;

FIG. 13: shows a schematic representation of a receiving body with closure elements;

FIG. 14: shows a schematic representation of a metering device of the system in a plan view in an open representation; and

FIG. 15: shows a schematic representation of a transport device of the system.

### DETAILED DESCRIPTION

In the drawings, reference numeral 100 denotes in its entirety a system in accordance with the present disclosure for processing pharmaceutical containers, which are designated by reference numeral 102. The containers can be, for example, vials, syringes, cartridges and/or ampules. The containers 102 can be stable or non-stable.

The system 100 is a preferred embodiment of the system in accordance with the present disclosure and makes possible the implementation of a preferred exemplary embodiment of the method in accordance with the present disclosure. Features of preferred exemplary embodiments of the method result from features of preferred embodiments of the system.

The system 100 serves to fill the containers 102 with a pharmaceutical product and then to close them. Production is carried out in accordance with a production plan, which includes specifications for the containers to be used, the product to be filled, the type of processing in production facilities and the hygiene requirements to be met in the process. In addition, the production plan can contain information relating in particular to the preparation for production.

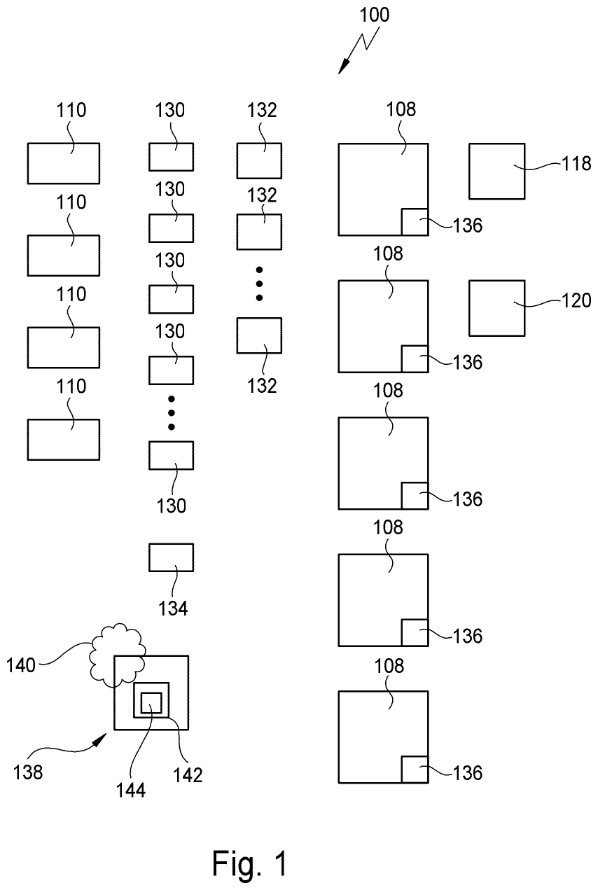

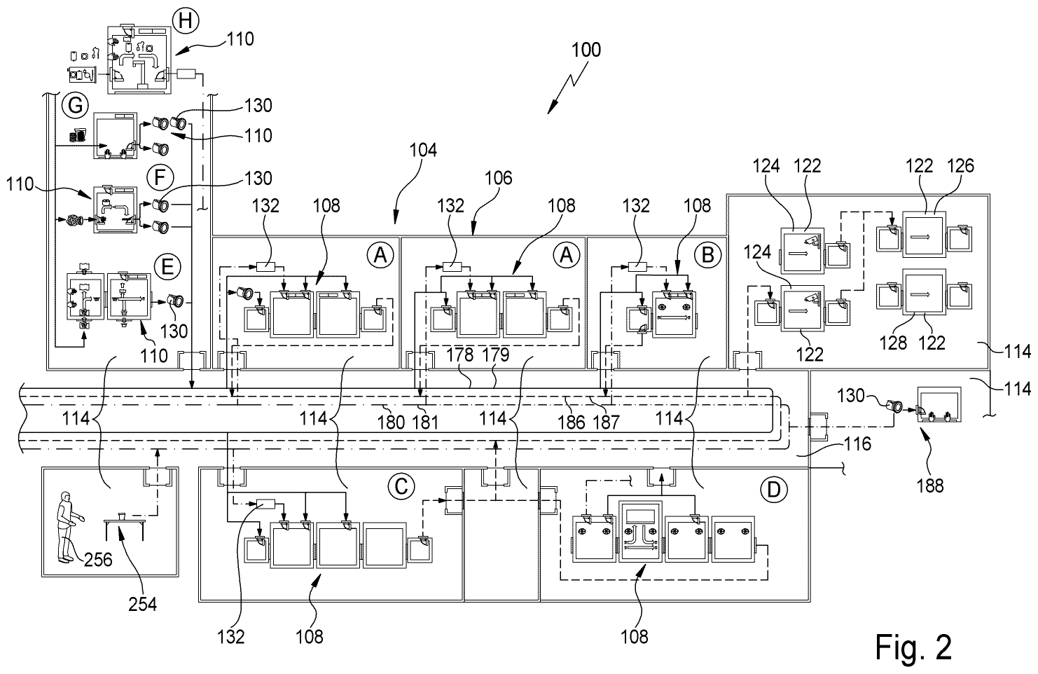

While FIG. 1 illustrates the system 100 in a schematic block diagram by way of example, FIG. 2 shows the system 100 in a layout in an environment 104 defined by a building 106. Different layouts of the system 100 are conceivable. The following description is not restrictive in this regard.

In the present example, the system 100 comprises a plurality of production facilities 108 for filling and closing the containers 102. In the present example, two identically configured production facilities 108 are provided, each marked with the letter “A”. Three further production facilities 108, which are different from the production facility 108A (“108+A”) and from each other, are marked “B”, “C” and “D” and are provided with reference numerals 108B, 108C and 108D. Unless reference is made below to a specific production facility, this is generally identified by reference numeral 108.

Accordingly, the system 100 comprises at least one preparation device 110. In the present case, a plurality of preparation devices are provided, which are marked “E”, “F”, “G” and “H” and are provided with reference numerals 110E, 110F, 110G and 110H. Where general reference is made below to only one preparation device, this is provided with reference numeral 110.

In the present exemplary embodiment, the environment 104 has a plurality of rooms 114 separated from one another by walls. The production facilities 108 are each located in their own rooms 114. The preparation devices 110 are arranged in the same room 114. The environment 104 can comprise a corridor 116 or the like to which the rooms 114 adjoin.

Overall, it can be advantageous if the preparation devices 110 are positioned spatially separate from the production facilities 108. More than just one production facility 108 can be arranged in a room 114, and/or more than just one preparation device 110 can be arranged in a room 114.

The system 100 preferably further comprises a sterilization device 118 positioned separately from the production facilities 108 and the preparation devices 110.

Furthermore, the system 100 can comprise a storage device 120 positioned separately from the production facilities 108 and the preparation devices 110.

The system 100 can further comprise at least one facility 122 for further processing of filled and closed containers 102. The facilities 122 can, for example, comprise an inspection facility 124, a labeling facility 126 and/or a processing facility 128 for further processing of filled and closed syringes.

The system 100 further comprises at least one container device 130, with a plurality of container devices 130 being provided in the present case.

Furthermore, the system 100 comprises at least one metering device 132, in this case a plurality of metering devices 132.

The system 100 further comprises at least one transport device 134, in this case favorably a plurality of transport devices 134.

The system 100 further comprises a control device 136 associated with respective production facility 108. The control device 136 is arranged, for example, at the location of the production facility 108 and comprised thereby. The control devices 136 can be coupled to each other for exchanging data.

A control device 136 can also be arranged on or comprised by one or more preparation devices 110.

Furthermore, the system 100 can comprise a data processing device 138 that can communicate with the control devices 136. The data processing device 138 can be formed by one or more servers and can be implemented, for example, via a cloud 140.

The data processing device 138 comprises a storage unit 142 in which, for example, the aforementioned production plan 144 is stored.

The data processing device 138 can comprise or form the control device(s) 136, or vice versa.

A respective production facility comprises a processing room 146. It is understood that the production facility 108 can comprise at least one chamber 148 with a wall arrangement 150 which encloses a processing room 146.

In the processing room 146, a respective filling station 152 is arranged at which the containers 102 are filled with the pharmaceutical product. In addition, a closing station 154 is arranged in each processing room 146. The containers 102 are closed by means of the closing station 154 with a closure element 156 provided for this purpose.

A controllable robotic handling device 158 is arranged in the processing room 146. The handling device 158 can be used in particular to handle the containers 102 and the closure elements 156. It is understood that more than one handling device 158 can be arranged in the respective processing room 146.

Upstream of the processing room 146, a production facility 108 can optionally comprise an in-coupling cell 160, which can be connected to the processing room 146 via a transfer opening 162. In the present example, this applies to the production facility 108A, 108B and 108C, but not to the production facility 108D, which in the present example does not comprise an in-coupling cell 160.

The production facility 108 comprises, at the processing room 146 and/or at the in-coupling cell 160, a connection device 164 for docking a corresponding container device 130 and a connection device 166 for docking a corresponding metering device 132. Containers 102 and closure elements 154 can be supplied to the processing room 146 directly or optionally indirectly via the in-coupling cell 160, in both cases via the handling device 158.

In the present example, the connection devices 164, 166 are configured in different ways. This ensures that the container device 130 and the metering device 132 are docked at the correct position of the production facility 108. The connection devices 164, 166 differ from each other, for example, in their size, but are otherwise configured to be functionally identical.

The connection devices 164, 166 are part of a respective connection system 168; in the present case an RTP (rapid transfer port) is used. For this purpose, the connection devices 164, 166 each comprise a closing element 170 for selectively opening or closing an insertion opening 172. This forms the alpha port of the RTP. Closing elements are shown schematically in the drawings in an open position.

A closing element 174 for selectively opening or closing a removal opening 176 is arranged on the respective container devices 130 or metering devices 132. This forms the beta port of the RTP.

Other types of connection systems or closing systems are conceivable.

In FIG. 2, a solid line 178 indicates a path 179 via which containers 102 and closure elements 156 can be supplied to the production facilities 108 by means of container devices 130 in the present example. Furthermore, via the path 179, for example, culture media, format parts and/or machine parts can be supplied for use in the production facilities 108.

A dot-dashed line 180 indicates a path 181. In the present example, the metering devices 132 are brought to the production facilities 108 via the path 181.

The production facility 108A is provided in the present case for processing vials and comprises a further processing room 182, which is connected to the processing room 146 via a transfer opening 162. In the processing room 146, the vials are filled and closed with supplied stoppers and then transferred to the processing room 182, in which a further controllable robotic handling device 158 is arranged. Closure elements 156 in the form of crimp caps are supplied by means of a further connection device 164 for container devices 130. The vials closed with stoppers are pharmaceutically sealed with the crimp caps in the processing room 182.

The production facilities 108A and 108C comprise, in the present case downstream of the processing room 182, an optional out-coupling cell 184 which is connected to the processing room 182 via a transfer opening 162. A connection device 164 for container devices 130 is provided on the out-coupling cell 184. Closed containers 102 can be supplied to the out-coupling cell from the processing room 146 by means of the handling device.

Filled and closed containers 102 can be coupled out via the out-coupling cell 184 and transferred to a container device 130. From there, the containers 102 can be supplied to further processing along a path 186, which is symbolized by a dashed line 187. For example, the containers are first supplied to the inspection facility 124 and then to the labeling facility 126, or in the case of syringes to the processing facility 128.

Used culture media, for example, are supplied to an analysis station 188 where they are analyzed.

The production facility 108B does not have a further processing room 182. In the production facility 108B syringes are processed which are pharmaceutically sealed by inserting a plunger stopper. Production material is supplied, for example, via the in-coupling cell 160, which can also be used as an out-coupling cell 184 for the filled and closed syringes.

In the present case, the production facility 108C is provided for processing vials containing a toxic product. In addition to the processing rooms 146, 182, a further processing room 190 is provided, which is located downstream of the processing room 182 and connected thereto via a transfer opening 162. In this case, the out-coupling cell 184 adjoins the processing room 190.

In the processing room 190, a cleaning station 192 is arranged for cleaning the outside of the pharmaceutically sealed vials. A handling device 158 is preferably also provided. Proceeding from the production facility 108C, the container devices 130 filled with the filled containers 102 can be supplied to further processing via a further room 114, which can be a washing room, for cleaning the outside of the container devices 130.

The production facility 108D comprises a further processing room 194, which is arranged between the processing rooms 146 and 182 and is connected to each of them via a transfer opening 162. A freeze-drying station 196 is arranged in the processing room 194 for processing the containers 102 by means of freeze-drying.

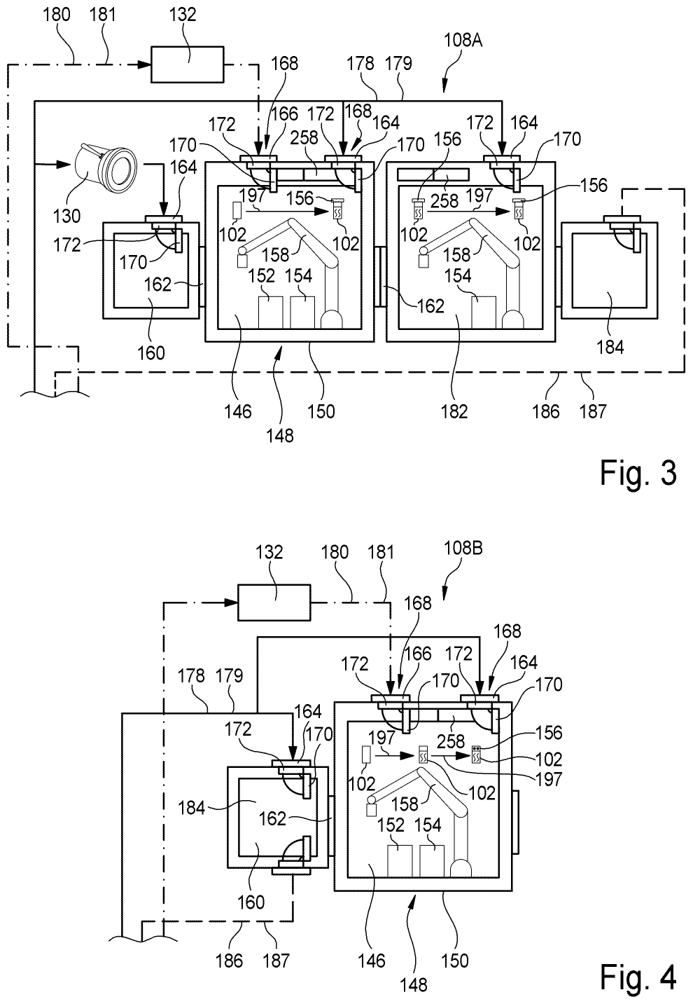

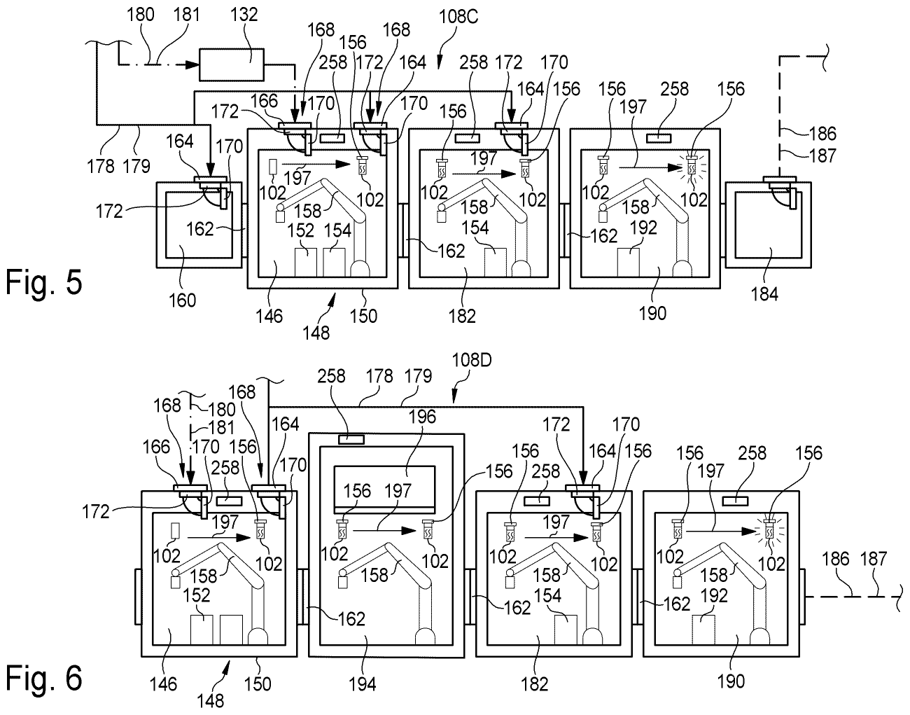

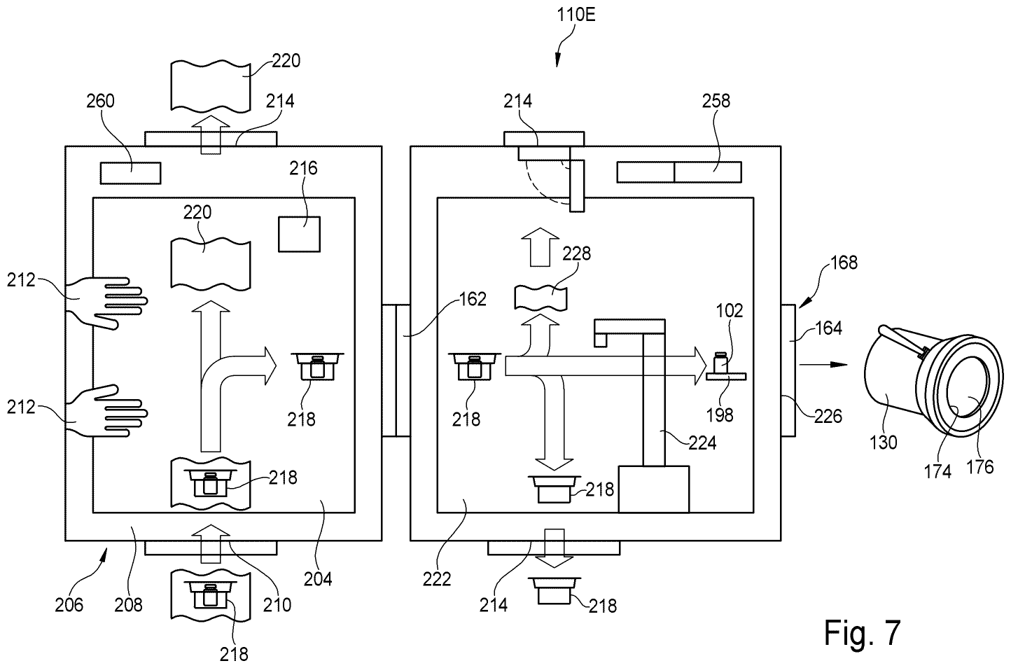

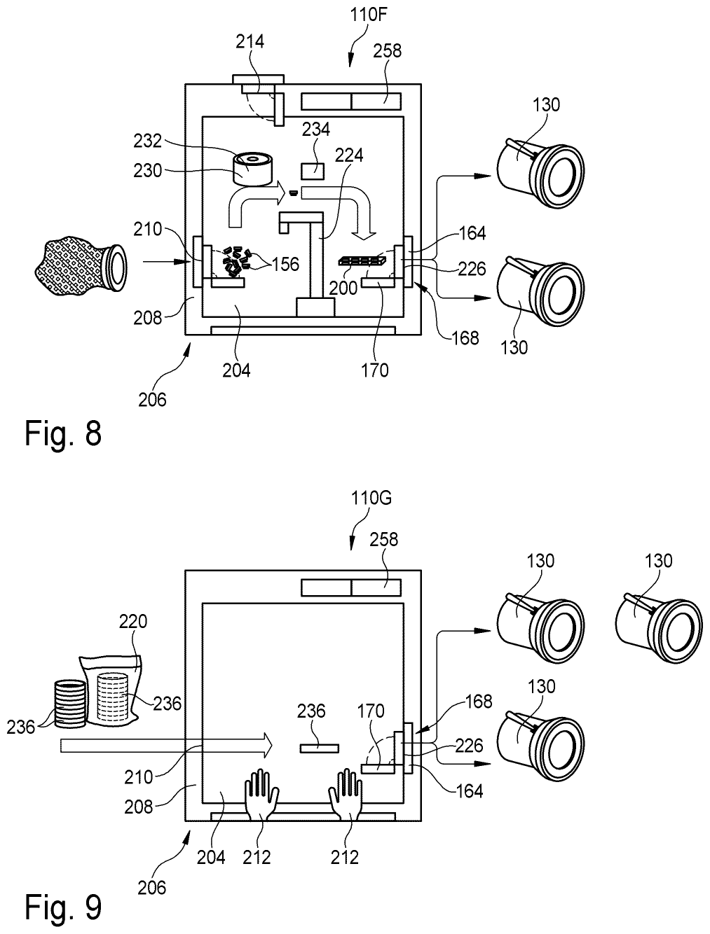

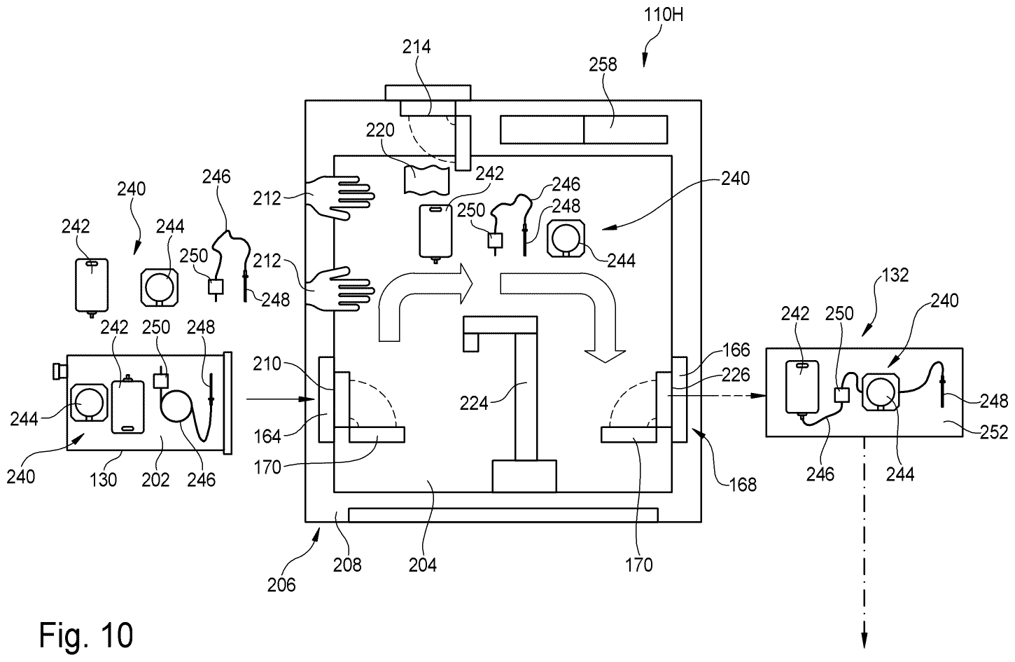

In FIGS. 3 to 6, the processing process for a respective container 102 is schematically illustrated at the top of the respective processing room, with a respective arrow 197: In the production facilities 108A, 108C, and 108D, in the processing room 146, the processing of the empty container 102 with filling and simple closure with caps, in the processing room 182, the pharmaceutical sealing with crimp caps; these are not shown separately; for the sake of clarity, the drawings symbolically show only one closure element 156. In the production facility 108B, the filling and pharmaceutical sealing is shown in a processing room 146. In the production facilities 108C and 108D, the cleaning of the outside of the pharmaceutically sealed container is shown in the processing room 190; in the production facility 108D, the freeze-drying of the not yet pharmaceutically sealed container 102 is additionally shown.

Favorably, the production facilities 108 in the processing rooms 146, 182, and 194 and in the optional in-coupling cell 160 each have, if present, a cleanliness class of type GMP A. Accordingly, the production facility 108 preferably has a continuous isolating property up to the processing room in which the containers are pharmaceutically sealed. In this case, the respective transfer openings 162 between Class A processing rooms or the Class A in-coupling cell 160 can, for example, be permanently open, as explained above.

A processing room adjoining the processing room 182 in which the containers are pharmaceutically sealed can have a lower cleanliness class, for example, type GMP C. In the production facilities 108C, 108D, this is the processing room 190. The same may apply to the optional out-coupling cell 184, unless it also serves as the in-coupling cell 160, as in the production facility 108B. In the case of type C rooms, a closing element with gas flow from the type A processing room or an airlock device, as explained above, is preferably arranged at the transfer openings 162.

However, it is conceivable that the processing room 190 and the optional out-coupling cell 184 also have a cleanliness class of type GMP A.

If a production facility 108 has a consistent cleanliness class throughout, in particular of type GMP A, in the processing room 146 and, if present, at least one further processing room and possibly an optional in-coupling cell 160 and/or out-coupling cell 184, it is possible in particular to implement a truly closed isolator device in the production facility 108, as described in Annex 1, “Manufacture of Sterile Medicinal Products” of August 2022 to the EU Guidelines for Good Manufacturing Practice (Annex 1 “Manufacture of Sterile Medicinal Products” to the “Guidelines for Good Manufacturing Practice for Medicinal Products for Human and Veterinary Use”).

Accordingly, the production facility 108 preferably comprises or forms a closed isolator device in accordance with Annex 1, “Manufacture of Sterile Medicinal Products”, to the EU Guidelines for Good Manufacturing Practice of August 2022.

In this case, it is possible in particular to supply production material and metering material via Class A container devices 130 and Class A metering devices 132, respectively, and to remove the filled and pharmaceutically sealed containers 102 via a Class A container device that is docked to the production facility 108 and to supply them for further processing, for example in one of the facilities 122.

In the system 100, the actual production in one of the production facilities 108 and the preparation of production in one or more of the preparation devices 110 are decoupled from each other and, in particular, spatially separated. This makes it possible to ensure, in the production facilities 108, the hygienic properties that are specified by production and stored, for example, in the production plan 144.

The decoupling of preparation and production allows the cleanliness properties to be maintained permanently, since the probability of disruption during production is reduced. Interruptions in the operation of the production facilities 108 are minimized, thereby increasing production throughput.

In particular, the system 100 proves to be advantageous for the processing of small or very small quantities, for example when filling toxic products and/or in the high-potent field.

In this context, it also proves to be advantageous that, due to the decoupling of preparation and production, the production facilities 108 require less space than conventional production facilities and systems.

The decoupling of preparation and production also proves to be advantageous for scaling and/or expanding the system 100. Depending on the requirements, for example, the number of preparation devices 110 can be adapted to the requirements of the production facilities 108, without for this purpose having to use complete units in which both the preparation and the production are carried out, as is the case with conventional systems.

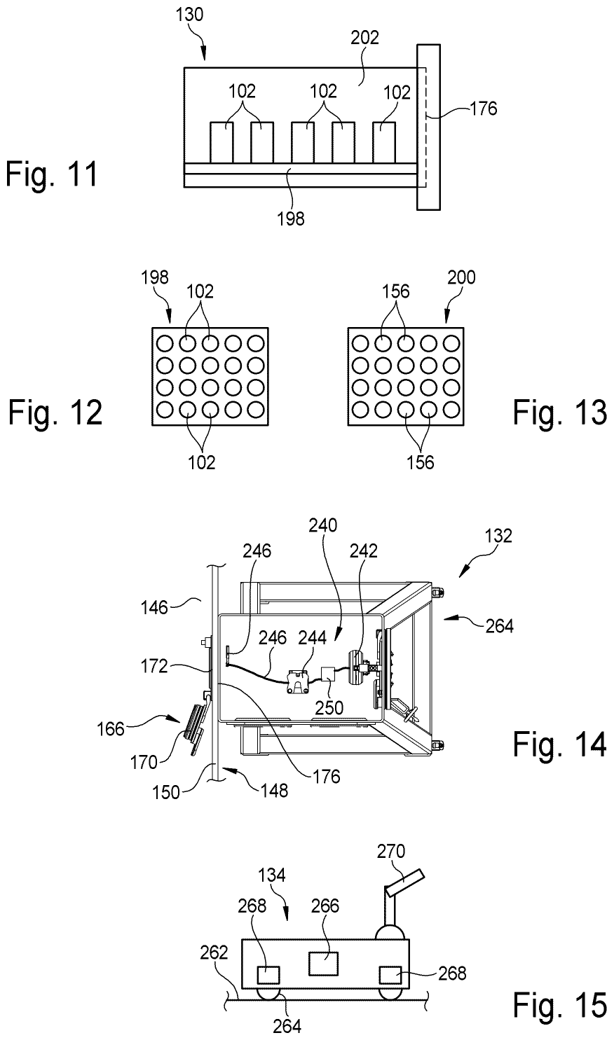

With regard to reliable and preferably unmanned processing in the production facilities 108, it is advantageous if the containers 102 are arranged in a predefined arrangement in receiving bodies 198. FIGS. 11 and 12 show, by way of example, such a receiving body 198 with a magazine-shaped design.

In a corresponding manner, the closure elements 156 in the present case are also arranged in a predefined arrangement in receiving bodies 200. This is shown schematically in FIG. 13 with a receiving body 200 in a magazine-shaped configuration.

Although the drawings show a plurality of containers 102 and closure elements 156 in the receiving body 198 and 200, respectively, it can be the case that only one container 102 or only one closure element 156 is received in each receiving body.

By means of the robotic handling devices 158, the receiving bodies 198, 200 supplied by means of the container devices 130 can be removed from the interiors 202 of the container devices 130 and transferred to the processing room 146, 182 or the in-coupling cell 160 for processing. Based on control data, the handling device 158 can supply the containers 102 to the filling station 152 and subsequently to the closing station 154. Based on control data, the handling device 158 can supply the closure elements 156 to the closing station 154. Required control data are stored, for example, in the production plan 144 and can be transmitted to the control device 136.

After filling and closing, the handling devices 158 can supply the receiving bodies 198, 200 back into the interiors 202 of docked container devices 130 for further processing.

The preparation devices 110 are provided for preparing production material.

A respective preparation device 110 comprises a work space 204. It is understood that the preparation device 110 can comprise a chamber 206 with a wall arrangement 208 that encloses one work space.

A connection device 164 for docking a container device 130 or, depending on the function, a connection device 166 for docking a metering device 132, is arranged on each preparation device 110. It is conceivable that both connection devices 164, 166 are provided.

In the present case, the preparation device 110E serves to prepare the containers 102. For this purpose, an insertion opening 210 is provided through which secondary packaging materials, for example tubs 218, with outer packaging 220 are inserted. Packaging can be removed by the user via a glove port 212 and disposed of via a disposal opening 214. At least one manual or automated disposal station 216 can be provided.

The preparation device 110 comprises in the present case the work space 204, in which the tubs 218 are freed from the outer packaging, as well as a further work space 222, which is connected to the work space 204 via a transfer opening 162.

The unpacked tubs 218 are transferred to the work space 222. This can be done manually by an operator or by a controllable robotic handling device 224 arranged in the work space 222.

A controllable robotic handling device 224 can also be arranged in the work space 204.

For example, the containers 102, which can be nested in the tub 218, are removed in the work space 222. The nest as a receiving body 198 or a separate receiving body 198 can be inserted into a docked container device 130 via a removal opening 226 in the connection device 164. Packaging material, for example the empty tub 218 or a cover film 228 of the tub 218, can be removed via further disposal openings 214.

It is understood that the system 100 can process more than just containers supplied via tubs 218. In general, however, the ability to process RTU (ready-to-use) production material proves to be advantageous.

The preparation device 110F is provided in this case to provide closure elements 156. The closure elements 156, which are supplied loosely, for example via a bag, are introduced into the work space 204 via the insertion opening 210. A work station 230 is preferably arranged in the work space 204. In the present example, the work station 230 comprises a sorting bowl 232. In general, the work station 230 preferably comprises a device to ensure a defined position and orientation of the closure elements. Advantageously, a handling device 224 is provided. An inspection station 234 can be provided, for example, to inspect the roundness of crimp caps.

The closure elements 156 are inserted into the receiving body 200 in a defined position and orientation and are placed into a docked container device 130 via the removal opening 226. Packaging can be disposed of via a disposal opening 214.

In the present example, the preparation device 110G is provided to provide culture media 236. For example, the culture media 236 are introduced into the work space 204 via a bag, another type of packaging or loosely through the insertion opening 210. The preparation device 110G preferably comprises at least one glove port 212, since it is possible to handle culture media 236 robotically to only a limited extent.

The culture media 236 can be placed loose or in a magazine through the removal opening 226 into a docked container device 130.

The preparation device 110H is provided here to set up the metering device 132. For this purpose, metering material 240 can be introduced into the work space 204. A glove port 212 and/or a handling device 224 can be provided.

The metering material 240 comprises, for example, a container 242, for example in the form of a bag, for the product, a metering unit 244 for conveying the product, a fluid conduit 246, a filling needle 248 arranged thereon and/or a filter element 250 in the form of a sterile filter in the fluid conduit 246. The metering unit 244 can, for example, be a pump unit.

The metering material 240 can be transferred into the receiving space 252 of the docked metering device 132.

During production, the metering device 132 can be docked to the connection device 166. By means of the handling device 158, the fluid conduit 246, at least partially, and the filling needle 248 can be introduced into the processing room 146 and arranged at the filling station 152 for filling.

Alternatively or additionally, for example, a holding part on which the filling needle 248 and/or the fluid conduit 246 are held and which is transferred from the receiving space 252 of the metering device 132 into the processing room 146, can form part of the filling station 152.

FIG. 2 schematically shows a preparation station 254. At the preparation station 254, a metering device 132 can be manually set up by a user 256 away from the preparation device 110H.

The respective preparation device 110 can have a required degree of cleanliness. For example, a respective work space has a cleanliness class of type GMP C or better. In particular, a cleanliness class of type GMP A can be provided.

To achieve a desired cleanliness class, the preparation device 110 can comprise or form a pharmaceutical isolator device 258. For example, in the present example, the preparation devices 110E (at the work space 222), 110F, 110G and 110H are isolator devices in which the GMP A cleanliness class prevails. The isolator device can be of ISO class 5.

In a corresponding manner, the respective production facility 108 can comprise or form a pharmaceutical isolator device 258, in particular of isolator class 5.

Alternatively or additionally, a decontamination device 260 can be provided, in particular for providing a decontamination atmosphere, for example by means of H₂O₂.

In the preparation device 110, the work space 204 can, for example, have a lower cleanliness class than the second work space 222. Decontamination takes place, for example, in the isolator device 258 at the work space 222.

If necessary, a terminal sterilization of the container devices 130 and/or the metering device 132 can be carried out at the external sterilization device 118 after filling in order to bring the contents of the respective devices 130, 132 to the intended cleanliness class.

The storage device 120 is provided for temporarily storing filled and closed container devices 130 and/or metering devices 132 after production and before use in the production facility 108. The required container devices 130 and/or metering devices 132 can be picked up from the storage device 120 as needed and transported to the relevant production facility 108.

FIG. 15 schematically shows the transport device 134. Said device 134 has a running gear 264 for moving on the set-down surface 262.

In principle, it is conceivable for the transport device 134 to be user-guided and to be used by the user 256 to transfer the container devices 130 and/or the metering devices 132.

Alternatively or additionally, the transport device 134 can preferably be configured to be self-propelled and self-steering and can be moved autonomously over the set-down surface 262. Accordingly, the transport device 134 is in particular a transfer robot which can comprise, for the purpose of moving, a control device 266 and a drive unit 268 for the running gear 264. The control device 266 is connected, for example, to the data processing device 138 and/or the control devices 136.

The transport device 134 can comprise a gripping element 270 by means of which the container device 130 and/or the metering device 132 can be releasably received, for example from the preparation device 110, the sterilization device 118 or the storage device 120, and with which they can be transferred to the production facility 108. It is understood that, after production, the further transport of the container device 130 can preferably be carried out by means of the transport device 134.

The metering device 132 can in turn have a running gear 264, as shown in FIG. 14, and is favorably also configured robotically, as has been described in connection with the transport device 134. In this case, a separate transport device 134 is not required to transport the metering device 132.

### LIST OF REFERENCE NUMERALS

100 System
102, 242 Container
104 Environment
106 Building
108 Production facility
110 Preparation device
114 Room
116 Corridor
118 Sterilization device
120 Storage device
122 Facility
124 Inspection facility
126 Labeling facility
128 Processing facility
130 Container device
132 Metering device
134 Transport device
136, 266 Control device
138 Data processing device
140 Cloud
142 Storage unit
144 Production plan
146, 182, 190, 194 Processing room
148, 206 Chamber
150, 208 Wall arrangement
152 Filling station
154 Closing station
156 Closure element
158, 224 Handling device
160 In-coupling cell
162 Transfer opening
164, 166 Connection device
168 Connection system
170, 174 Closing element
172, 210 Insertion opening
176, 226 Removal opening
178 Solid line
179, 181, 186 Path
180 Dash-dotted line
184 Out-coupling cell
187 Dashed line
188 Analysis station
192 Cleaning station
196 Freeze-drying station
197 Arrow
198, 200 Receiving body
202 Interior
204, 222 Work space
206 Chamber
212 Glove port
214 Disposal opening
216 Disposal station
218 Tub
220 Outer packaging
228 Cover film
230 Work station
232 Sorting bowl
234 Inspection station
236 Culture medium
240 Metering material
244 Metering unit
246 Fluid conduit
248 Filling needle
250 Filter element
252 Receiving space
254 Preparation station
256 User
258 Isolator device
260 Decontamination device
262 Set-down surface
264 Running gear
268 Drive unit
270 Gripping element

1. A system for processing pharmaceutical containers, comprising:
at least one production facility for filling and closing the containers, which comprises, in at least one processing room, a filling station for filling the containers with a pharmaceutical product and a closing station for closing the containers with closure elements,
wherein the at least one production facility comprises a controllable robotic handling device which is arranged in the at least one processing room, is configured for handling pharmaceutical production material comprising containers and/or closure elements received in a predefined arrangement in receiving bodies, and which comprises or forms at least one connection device for docking an external transportable container device and/or an external transportable metering device;
at least one preparation device which is or is arrangeable spatially remote from the at least one production facility and comprises or forms at least one work space and at least one connection device for docking the container device, wherein pharmaceutical production material is introducible into the at least one work space, which production material comprises containers to be processed and/or closure elements, wherein the containers and/or the closure elements are insertable into receiving bodies in a predefined arrangement in the at least one work space;
at least one transportable container device, by means of which the containers received in the receiving body and/or the closure elements received in the receiving body are bringable directly or indirectly from the at least one preparation device to at least one production facility and, when the container device is docked to the at least one production facility, are introducible into the at least one processing room by means of the handling device;
at least one transportable metering device comprising, in a receiving space, pharmaceutical metering material comprising a fluid conduit and a filling needle arranged thereon, which is introducible into the at least one processing room by means of the handling device when the metering device is docked to the at least one production facility.

2. The system in accordance with claim 1,
wherein
the system comprises at least one control device for controlling the at least one production facility and/or the at least one preparation device.

3. The system in accordance with claim 1,
wherein
a cleanliness class of the at least one processing room in accordance with type GMP A and/or by a cleanliness class of the at least one work space in accordance with type GMP C or better, for example in accordance with type GMP A, is provided.

4. The system in accordance with claim 1,
wherein
the system comprises a separate sterilization device, in particular an autoclave device, for sterilizing objects received in the at least one container device and/or the at least one metering device.

5. The system in accordance with claim 1,
wherein
at least one of the following applies:
the at least one preparation device comprises an insertion opening via which the production material is introducible into the work space;
the at least one preparation device comprises a removal opening in the connection device, via which the production material is transferable into the container device;
the at least one preparation device comprises a disposal opening for waste;
the at least one preparation device comprises or forms a pharmaceutical isolator device.

6. The system in accordance with claim 1,
wherein
the at least one preparation device comprises or forms a decontamination device for providing a defined decontamination atmosphere in the at least one work space, in particular by means of H₂O₂, and/or a connecting element for connecting an external decontamination device to the preparation device.

7. The system in accordance with claim 1,
wherein
at least one of the following applies:
the system comprises two or more preparation devices;
the system comprises a preparation device for providing the containers and a preparation device separate therefrom for providing closure elements;
the system comprises a preparation device for providing culture media for introduction into the at least one production facility via the at least one container device, which is preferably separate from a production device for providing containers and/or closure elements;
the system comprises a preparation device for providing format parts and/or machine parts for introduction into the at least one production facility via the at least one container device, which is preferably separate from a production device for providing containers and/or closure elements;
the system comprises a preparation device for providing the metering material in the work space, it being possible for the at least one metering device to be docked to the preparation device and the metering material being transferable into the receiving space.

8. The system in accordance with claim 1,
wherein
the at least one preparation device comprises two separate work spaces which are connected to one another via a transfer opening, wherein production material is prepared, in particular unpacked, sterilized and/or decontaminated, in one work space and is introduced into the second work space via the transfer opening, it being possible for the at least one container device to dock in the region of the second work space.

9. The system in accordance with claim 1,
wherein
the at least one preparation device comprises a controllable robotic handling device in the at least one work space for moving the production material and/or wherein the at least one preparation device has at least one glove port for a user into the at least one work space.

10. The system in accordance with claim 1,
wherein
the system comprises, spatially separated from the at least one production facility and the at least one preparation device, a storage device for temporarily storing filled container devices and/or metering devices after filling at the at least one preparation device and before use at the at least one production facility.

11. The system in accordance with claim 1,
wherein
the at least one metering device comprises at least one of the following, preferably in the receiving space:
the fluid conduit and the filling needle;
a container for receiving the product to be filled;
a metering unit for conveying the product through the fluid conduit;
a filter element connected into the fluid conduit.

12. The system in accordance with claim 1,
wherein
the system comprises two or more production facilities.

13. The system in accordance with claim 1,
wherein
the at least one production facility comprises two connection devices which are preferably configured differently from one another, it being possible for the at least one container device to be docked to one connection device and for the at least one metering device to be docked to the other connection device.

14. The system in accordance with claim 1,
wherein
the filling station and the closing station are arranged in the same processing room of the at least one production facility.

15. The system in accordance with claim 1,
wherein
at least one of the following applies:
the at least one production facility comprises a further processing room and a further closing station, which is arranged in the further processing room, the processing rooms being connected to one another via a transfer opening;
the at least one production facility comprises a further processing room having a cleaning station, which are preferably connected to the at least one processing room via a transfer opening;
the at least one production facility comprises a further processing room having a freeze-drying station, which are preferably connected to the at least one processing room via a transfer opening.

16. The system in accordance with claim 1,
wherein
the at least one production facility is designed to be free of a glove port for the user.

17. The system in accordance with claim 1,
wherein
the system comprises at least one transport device by means of which the container device is received or releasably receivable, and/or wherein the system comprises at least one transport device by means of which the metering device is received or releasably receivable, wherein the at least one transport device comprises a running gear for moving on a set-down surface.

18. The system in accordance with claim 17,
wherein
the at least one transport device is configured to be self-propelled and self-steering for autonomous transport of the at least one container device or the at least one metering device.

19. The system in accordance with claim 1,
wherein
the system comprises a data processing device having a storage unit in which a production plan is stored, wherein at least one of the following is controllable by the data processing device indirectly or indirectly based on the production plan: the at least one production facility, the at least one preparation device, the at least one transport device.

20. A method for operating a system in accordance with claim 1, comprising:
docking at least one container device to the at least one production facility;
introducing into the processing room, by means of the at least one handling device, a receiving body which receives at least one container to be filled in a predefined arrangement;
introducing into the processing room, by means of the at least one handling device, a receiving body which receives at least one closure element in a predefined arrangement;
docking at least one metering device to the at least one production facility;
introducing the filling needle and, at least partially, the fluid conduit into the at least one processing room;
filling and closing the at least one container.
